(19) United States
(12) Patent Application Publication
(10) Pub. No.: US 2011/0066162 A1
(43) Pub. Date: Mar. 17, 2011

(54) CRYO-MICRO-DERMABRASION

(75) Inventor: Donald Cohen, Irvine, CA (US)

(73) Assignee: Vandolay, Inc., Carlsbad, CA (US)

(21) Appl. No.: 12/561,055

(22) Filed: Sep. 16, 2009

(51) Int. Cl. A61B 18/02

(52) U.S. Cl. 606/131

(57) ABSTRACT

The invention is a device that generates a cool fluid stream that has properties adequate to remove superficial layers of tissue. The stream is generated to render the target tissue much more brittle in its mechanical behavior than it would otherwise be; principally by temperature and pressure rise and decay rates. The primary intended use is for dermatologic skin resurfacing, though it may be used to remove superficial layers of tissue anywhere in the body.

### FIELD OF THE INVENTION

The field of the invention is tissue removal, with principal application of dermatological skin resurfacing.

### BACKGROUND

Many people seek skin treatment to improve the appearance of their skin. There are several procedures in which the outer layers of the skin are removed/eroded. After the subsequent healing process, new skin proliferates and the appearance of the skin is generally improved, smoother and more uniform in hue.

A distinction can be made between two of theses procedures: (i) Dermabrasion and (ii) Microdermabrasion. Dermabrasion is more aggressive; it is a medical procedure typically performed by a surgeon. Generally it involves the erosion or “peeling” of skin while the patient is under the influence of anesthesia. It can employ high speed rotating abrasive sanding machines on the skin while the skin is chilled.

Microdermabrasion in contrast is like sandblasting. Abrasive particles are accelerated to impact with the skin. Though not as aggressive, it works well to improve skin imperfections such as fine lines, dull skin, mild acne scars, and age spots. It works on all skin types though it works best on the superficial skin conditions. Healing time is much quicker than for dermabrasion.

For either of these procedures, the cosmetic result tends to be best when the applied treatment is uniform. The skin then heals and generates new tissue uniformly creating a desirable result.

Other related procedures include chemical peeling (in which a caustic substance is used), laser peeling (in which laser radiation is used) to elicit the subsequent skin healing.

Another skin cutting procedure is described in (WO/1999/037229) EPIDERMAL AND DERMAL SKIN REMOVAL APPARATUS. In this Coleman, et. al. disclose the use of high pressure water stream to cut into skin. The patent also cites example embodiments including mixing air in with the water to create “particlization” of the water stream.

For complete understanding of the current invention, it is important to understand that skin is a viscoelastic material. In other words, unlike typical engineering materials such as steel, the mechanical behavior (stress and strain) of skin is time dependent. The most pertinent of these time dependent behaviors is that skin becomes more rigid and more brittle at high loading rates and at low temperatures. This is illustrated graphically in FIGS. 1a b and c (borrowed from scudc) which shows how plastics (like skin) behave viscoelastically. When the skin behavior is brittle, it can require much less energy to fracture.

Another procedure to improve skin appearance involves creating small regularly spaced holes in the skin. The subsequent healing process tends to make the skin appear tighter. The holes have been made with laser radiation or with sharp needles or with electric current. The technique is called fractional skin treatment or tightening.

Other tissue treatments include cataractous lens removal prior to intraocular lens implant, liposuction and tumor and cyst resections. These can be done with cutting implements. Ultrasonically vibrated aspiration needles or water jet s are often used to accelerate the procedures.

### Limitations of Present Technologies

Widespread use of dermabrasion is limited by the usual limitations of surgical procedures. It can be expensive; it has the usual surgical risks; it can take a considerable time to heal; healing can be painful for a protracted time. In addition, cosmetically the appearance is not desirable until healing has progressed substantially. The treatment can be non-uniform. It requires training and expertise to create a uniform injury and ultimately a uniform result. The treatment also causes sun sensitivity during the healing process.

Microdermabrasion reduces some of these limitations, and while it works best on superficial skin conditions it does not work as well on the deeper ones. Additional drawbacks include some pain and the nuisance of the abrasive material. This can be an annoyance to the patient—who continues to clean the remnants of the procedure for days afterwards. It can pose an inhalation hazard as well. If the treatment is inadvertently allowed to progress too deeply, the foreign material may illicit undesirable effects. It also contributes to the challenge of keeping the equipment and procedure area clean for subsequent patients and staff. Also, Microdermabrasion mandates uniform application by a trained operator to achieve desirable results. Non-uniformity of treatment can cause unattractive results.

Results of these procedures can vary—not only from one patient to another, but from spot to spot on the skin of a single patient. Non-uniformity in the application of the treatment can thus produce non-uniform results.

While Coleman, et. al. disclose the use of high pressure water stream to cut into skin, they imply that the cutting action is attributable to the use of high pressure water stream. It does not anticipate (1) using a cryo liquid propellant, (2) achieving the desired tissue failure (cutting) goal at substantially lower pressure by straining the tissue at a high strain rate at which the tissue is more brittle and (3) achieving the desired tissue failure goal at substantially lower pressure by straining the tissue at a low temperature at which the tissue is more brittle.

With respect to tissue resection, liposuction and cataractous lens removal, it can be difficult to remove only the target tissue without also inadvertently damaging neighboring tissues.

It is thus an objective of the current invention to provide skin resurfacing at less cost, shorter healing time and less risk than dermabrasion.

It is another objective of the current invention to provide very uniform treatment, especially in comparison to manually guided microdermabrasion.

It is another objective of the current invention to provide treatment while causing less pain than prevailing procedures cause.

In a particular embodiment of the current invention it is another objective to provide treatment without embedding particulate matter in the flesh of the patient.

It is an objective of the current invention to provide skin resurfacing by the use of pulsed fluid that renders the target skin much more brittle in its mechanical behavior than it would otherwise be.

In a particular embodiment of the current invention it is another objective to achieve liquid stream induced skin fracture with lower pressure than used by prior art liquid stream cutting devices.

In another embodiment of the current invention it is another objective to semi-automatically treat age spots and other skin blemishes.

In another embodiment of the current invention it is another objective to provide fractional treatment of the skin with the goal of skin tightening.

In another embodiment of the current invention it is another objective to remove tissue controllably anywhere in the body, such as cataractous lens removal, liposuction or tumor resection.

### ADDITIONAL PRIOR ART INFORMATION

### Patent Weblinks

http://www.freepatentsonline.com/6726693.html

http://www.freepatentsonline.com/6306119.html

http://www.freepatentsonline.com/6226996.html

http://www.freepatentsonline.com/6174225.html

http://www.freepatentsonline.com/5913711.html

http://www.freepatentsonline.com/4703590.html

http://www.freepatentsonline.com/5037432.html

http://www.freepatentsonline.com/4378804.html

http://www.freepatentsonline.com/5100412.html

http://www.freepatentsonline.com/5207234.html

http://www.freepatentsonline.com/5810842.html

WO/1999/037229

20070149991

20050283141

20040220512

20060173518

20070232966

7001355

20040010222

20060122508

20070179481

20060047281

### Literature Weblinks

http://matse1.mse.uiuc.edu/ceramics/prin.html

http://www.scudc.scu.edu/cmdoc/dg_doc/develop/material/property/a2200002.htm

http://www.aquamationinc.com/

http://www.environ.co.za/contents/products/roll_cit/roll_cit_medical.htm

http://www.vivida.co.za/med_treat

http://www.amo-inc.com/products/cataract-implants/phacoemulsification-systems/whitestar-signature-phacoemulsification-system

http://www.infinitivision.com/energy/energy-aqualase.html

### SUMMARY OF THE INVENTION

The current invention may be summarized as a device that generates a cool fluid stream that has properties adequate to remove superficial layers of tissue. The stream is generated to render the target skin much more brittle in its mechanical behavior than it would otherwise be. Many variations may be contemplated. Details of some of the variations contemplated follow.

### BRIEF DESCRIPTION OF THE DRAWINGS

FIG. 1a is a stress-strain diagram showing how a brittle material is stiff and elastic up until the point at which it fractures.

FIG. 1b is a stress-strain diagram showing a ductile material has an elastic range, then a plastic range which allows it to stretch and absorb energy up until the point at which it fractures.

FIG. 1c is a stress-strain diagram showing how viscoelastic materials become more brittle at low temperature or high strain rate.

FIG. 2 shows an idealized dynamic trace of pressure applied to the skin by cold fluid over the course of time, and simultaneously the first derivative of the pressure with respect to time on the same time line. The pressure trace is shown at the top—with the pressure magnitude scale shown on the left vertical axis. The pressure derivative (dP/dt) is shown in the lower trace with the pressure rate scale shown on the right vertical axis.

FIG. 3 is a photographic representation of a series of fluid pulses separated by air.

FIG. 4a is an isometric depiction of the distal tip of an embodiment of the instrument of the current invention.

FIG. 4b is an isometric depiction of the distal tip of the instrument of FIG. 4a, shown in partial section.

FIG. 5a is an isometric depiction of the distal tip of an embodiment of the instrument, shown in partial section illustrating how tissue can be drawn into the aspiration channel by application of partial vacuum.

FIG. 5b is the isometric partial section of the instrument of FIG. 5a, further depicting fluid pulses being injected against the superficial skin tissue drawn in through the aspiration slots.

FIG. 5c is the isometric partial section of the instrument of FIG. 5b, further depicting the remnants of fluid pulses vapor and skin debris being drawn into the proximal aspiration channel.

FIG. 6 illustrates an alternative embodiment in which the injection pulses are directed perpendicularly at the tissue, though the aspiration and injection functions are otherwise unchanged.

FIG. 7a is a photograph of an XY scanner cold fluid pulse system shown partially disassembled.

FIG. 7b is a depiction of an XY scanner cold fluid pulse system shown partially disassembled.

FIG. 8 is a depiction of a heating system that is focused on the same skin target as the cold stream. It includes an infrared heat source, an elliptical reflector, a flat reflector with a centrally located hole through which the cold stream nozzle is passed.

FIG. 9 depicts a view of part of an embodiment including aspiration pump, tubing and trap, pressurized cryo fluid, solenoid valve, injector nozzle, surrounding aspiration paths, shallow skin treatment trough and z-direction spring loaded follower shown in partial section.

### DETAILED DESCRIPTION OF THE INVENTION

Human skin is a viscoelastic material. This means that the unlike elastic materials, stress and strain are time and temperature dependent. As one example of the time dependence—if a fixed stress (load) were placed on an elastic material (such as steel), a particular strain (deflection) would result; and the strain would remain constant as long as the stress were held constant. Because of the viscoelasticity of skin however, a constant stress results in a strain that continues to change with time.

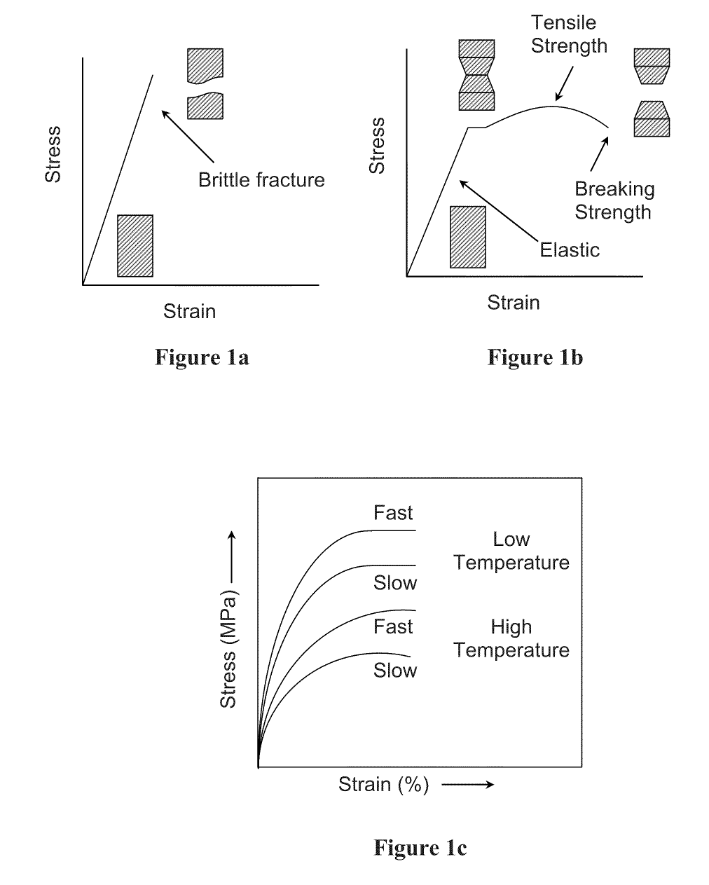

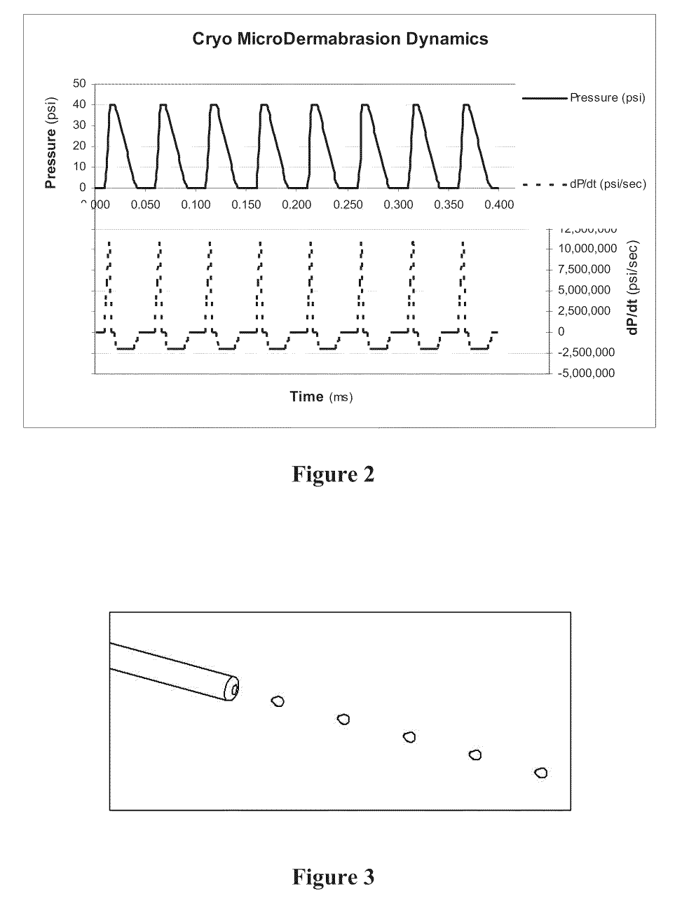

Also, the rate at which strain is applied to the tissue affects its mechanical response. When the skin is strained at a fast rate, it behaves more like a rigid, brittle material as illustrated in FIG. 1c.

FIGS. 1a and 1b (copied from http://matse1.mse.uiuc.edu/ceramics/prin.html) illustrate that when a material is brittle, it generally requires less energy to fracture it compared to when it is ductile. When it is brittle, it breaks at a much lower strain—i.e. deformation. The energy required to fracture the material (integrated area under the stress strain curve) is much lower for the brittle material. It requires less energy and less strain to fracture the skin when it is brittle. It is easier to treat the skin when the temperature is cold and the strain rate is high.

When the strain rate is low, the skin tends to seem less stiff and is able to stretch much more before it ultimately breaks. This implies that the skin can be stressed to its limit at a lower value as long as it is strained more quickly. In other words, it is easier to break portions of the skin free from its attachments using fast pulses rather than if slowly applied.

Similarly, skin behaves more brittle at low temperature than at ambient temperature. It is easier to break off fragments of skin at lower temperature.

Fluid

In a preferred embodiment the fluid stream is pressurized liquefied gas such as liquid nitrogen or liquid carbon dioxide or refrigerant such as R134-A tetrafluoroethane. Ideally the liquefied gas is biocompatible. At a minimum, it is benign. It should not be an explosive, flammable or inhalation hazard. Ideally it contains no abrasive pervasive particulate to cause problems.

An acceptable alternative is a pressurized chilled liquid, such as saline. Higher than usual salt concentration may be used to permit freezing point depression and so a lower temperature liquid.

Temperature

The temperature of the propelled fluid as it interacts with the skin is preferably below 5° C. so that the local target skin temperature is reduced so that the mechanical properties are more brittle, i.e. amenable to fracture compared to the properties at ambient temperature.

To further increase the brittle behavior of the tissue, it is desirable to create a rapid change of temperature. Because most materials expand at high temperatures and shrink at low temperatures—changes in temperature create stresses in materials. When this change is rapid, the material exhibits more brittle behavior. Just as a cold drinking glass may crack when filled with a hot beverage, and just as a hot engine block may crack when filled with cold water; rapidly cooled (or rapidly heated) tissue may crack in response to the stress.

The rapid heating between cold pulses may be accomplished by any of several ways. One way is to apply heat pulses. The source of energy for the heat pulses may be electromagnetic (as from a laser, a lamp or a radiant heater), acoustic (as from an ultrasound vibration) or other. Another way is to apply a continuous flow of heat (as from a radiant heater) wherein the heat flow to the tissue is effectively interrupted by pulses of cold fluid. Yet another way to achieve the rapid heating between cold pulses is to apply pulses of hot substance between pulses of the cold fluid.

It is desirable to create a rate of change of temperature of the material contacting the target tissue of at least 10° C./second, though a thermal rate of change of greater than 500° C./second is more desirable. The duration of a typical pulse is considerably shorter than 1 second—so the rate of change of temperature can be high even though the total change in temperature may be modest.

Pressure Dynamics

The pressure of the chilled stream is closely related to the maximum stress experienced by the skin tissue. Maintaining a steady fluid pressure stream below the ultimate strength of the skin will do little to remove tissue. On the other hand, applying the same pressure in a pulsatile manner can rapidly fracture and remove tissue. The key determining factor in this regard is the rate of change at which pressure is applied and terminated. In an embodiment of the present invention, the minimum rate of change of pressure rise is 100 psi/second and the minimum rate of change of pressure decay to elicit the desired rate of strain is −100 psi/second. Higher rates of pressure change promote efficacy and are preferred.

There is no minimum pulse duration, though from a practical standpoint, the preferred minimum pulse duration is 1 millisecond.

There is no minimum pulse repetition rate, though higher repetition rate will provide higher tissue removal efficiency. From a practical standpoint, the preferred minimum pulse repetition rate is 4 Hz.

There is no minimum pulse pressure, though from a practical standpoint, the preferred mean pulse pressure is 30 psi.

In order to achieve the desired pulse pressure rise and decay rates, it is important to minimize system mechanical compliance.

Particulate

In the preferred embodiment, there are no conventional abrasive solid particulate inclusions in the fluid stream. Particulates of a different nature may be included. These include (i) particles that are solid only at cold temperature, (ii) liquid pulse “particles” separated by pulses of vapor or (iii) water soluble particles.

In other embodiments, abrasive particulate inclusion is desired—it is preferred that the particles leave no debris. Particles that leave no debris would include water ice particles, dry ice particles or equivalent—similar to those in U.S. Pat. No. 6,306,119.

In another embodiment, minute aliquots of gas would be injected or interspersed with the liquid. The effect of the impact of a gas/liquid interface against the skin could be the equivalent of the high rise rate pulse previously described herein. The included gas could be air, nitrogen, CO₂ or equivalent. The resulting stream of gas and liquid might appear as in FIG. 9, borrowed from aquamotion. The gas should have low solubility in the liquid.

In another embodiment particulates may be included that are biocompatible and soluble in water but insoluble in the cold liquid stream. Solutes may include salts, such as NaCl, KCl, sulfates, phosphates, etc, amino acids, small proteins, sugars, carbohydrates or even fats.

In other embodiments, it is acceptable to have conventional abrasive inclusions, such as Al₂O₃, SiO₂ or equivalent.

Scanner

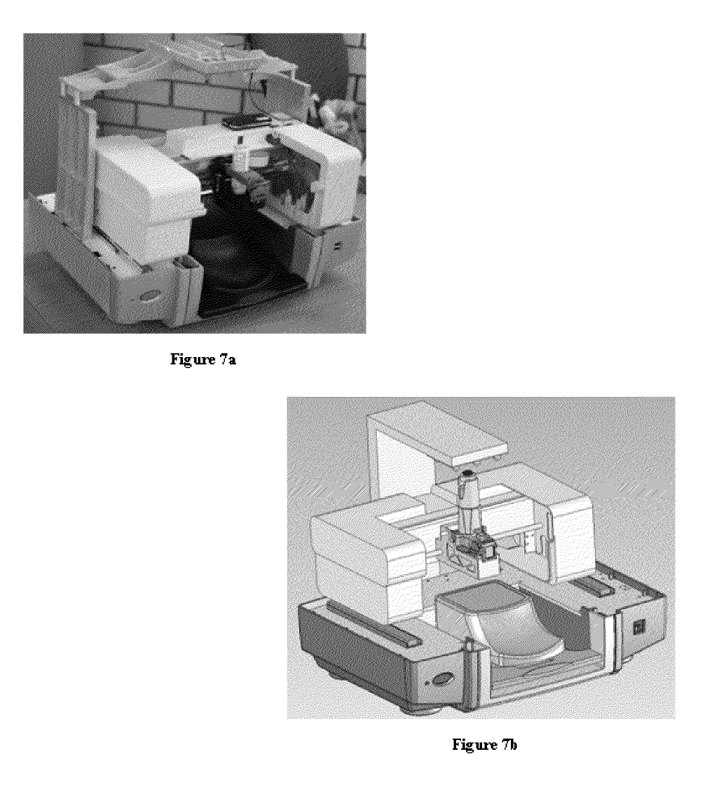

The cold erosive spray could be applied to the skin via a handpiece and scanned manually to treat an area larger than the spray area. In a preferred embodiment the scanning is accomplished via the use of an automatic motion system. The XY control of spray may be manual. Alternatively the XY control of spray may be semi-automatic—as with the aid of a camera to view the skin area, and a mouse to select the treatment areas. It is preferred that the actual scanning is accomplished with the guidance of an automatic system. A preferred scanner is an XY electromechanical manipulator, such as depicted in FIGS. 7a and 7b. It is important that the nozzle is selected so that the stream remains relatively coherent (collimated) from the nozzle outlet to the interface with the skin. In this way, the skin receives basically the same treatment irrespective of its distance from the nozzle outlet.

FIGS. 7a and b illustrate an embodiment of a scanner system that further include an illumination and visualization system to plan and monitor the procedure.

In yet another embodiment, the distance from the nozzle outlet to the skin is maintained at a nearly constant gap. The height can be automatically measured and kept constant. Methods of adjusting gap include sonar, angled beams which optically indicate height by measuring the distance between the beams on a single camera image, electro-mechanical sensor that changes impedance between two or more electrodes when they contact the skin, etc.

Alternatively, the height can be mechanically adjusted, i.e. a spring loaded nozzle that rolls over the skin surface. With the roller or rollers contacting the skin, the adjacent nozzle will also be at a nearly constant distance from the target skin despite undulations in skin topography.

Nozzle

The nozzle is chosen to allow for a nearly collimated coherent spray over approximately a 1 inch distance so that the diameter of the pulse is unaffected by the gap between the nozzle outlet and the skin. This includes choice of materials, surface finish (friction factor with the pressurized cool fluid), pressure, density, orifice size, length (Reynold's Number), flow direction changes, flow area changes and tapers.

The design of the nozzle may also include venting to atmosphere to avoid disturbing the stream coherence when the valve is opened or closed. The vent may include a check valve.

The nozzle may also include multiple orifices with actively controlled valves so that gas may be alternated with the fluid stream pulses.

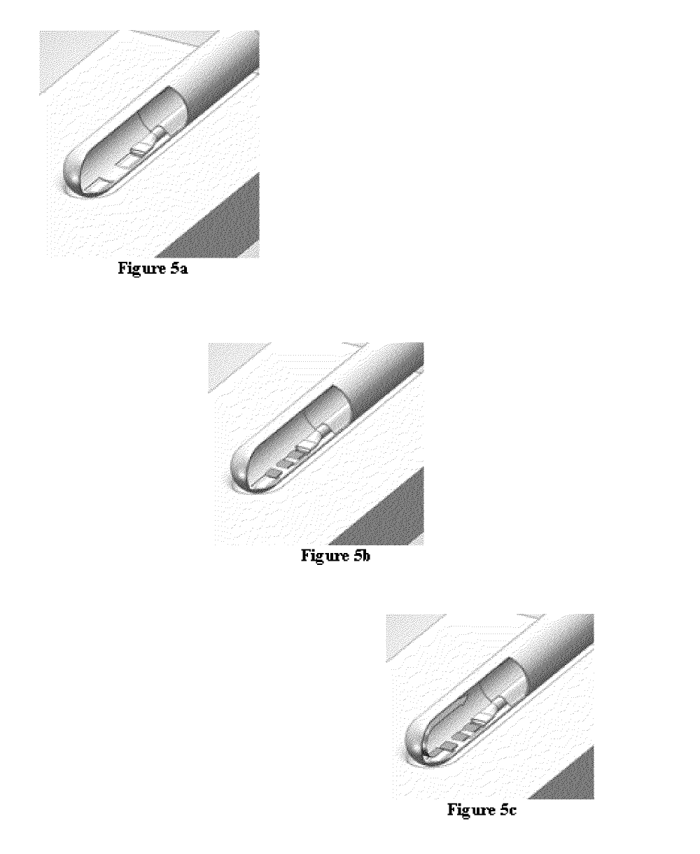

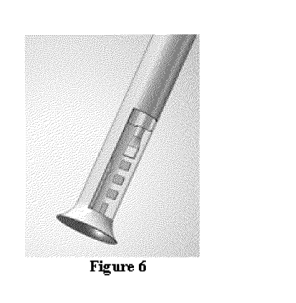

The nozzle must be directed at the superficial layers of skin so that it can chill the skin and rapidly strain it to the fracture point. The orientation can be orthogonal, tangential, or anywhere in between. FIG. 5a b and c illustrate how the target tissue may be drawn into the path of the nozzle injector to fracture by shear the portion of skin to be treated, and is then drawn back into the aspiration channel. FIG. 6 illustrates the same for a perpendicular orientation.

It is important that the stream size does not enlarge greatly before it interacts with the tissue; that would reduce the stress and impede the ability of the stream to erode tissue. It can be very desirable to have the cutting potential of the stream reduced quickly with distance away from the outlet. With such a spray, only the tissue close to the nozzle outlet will be subject to fracture. The tissue further away could only be subjected to a larger spray and thus a lower stress; thus tissue that is not in the immediate vicinity of the nozzle outlet would not be inadvertently fractured or damaged.

Valving

There are many ways to created high strain rate pulses. One way to accomplish this is to use fast solenoid valves. Some solenoid valves are rated to handle relatively high pressures at relatively fast rates. Solenoids can switch the fluid channel from totally closed to totally open in just a few milliseconds.

Piezo valves generally are not rated to handle pressures as high as solenoid valves do, yet they can often switch from closed to open and back to closed again even faster than solenoid valves. Consequently it is possible for piezo valves to achieve pressure rise and decay rates as fast as or faster than solenoid vales.

Alternatively, other mechanical choppers can create high pressure rise and decay rates without ever completely reducing the flow to zero. For example a gating wheel can interrupt a continuous spray to chop a coherent stream into pulses. Other similar mechanisms can achieve similarly good pulse formations.

Heater

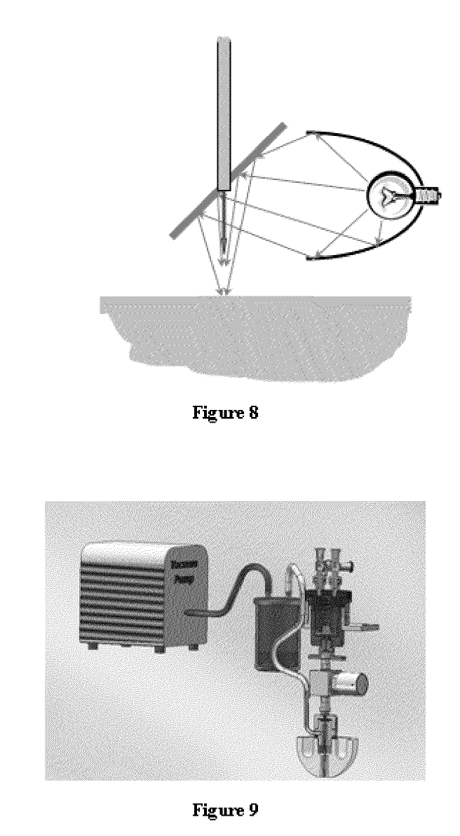

Though the cold temperature will serve to mitigate some of the pain of tissue disruption, the extreme cold can also be uncomfortable—so a heater may be provided to immediately follow the cryo pulses. The heat is preferably confocal with the cryo spray. In one embodiment the heater would be a tungsten filament of a halogen bulb focused by an elliptical reflector to the size of the spray on the same spot. This may be accomplished with the use of a second reflection element—a planar reflector with a hole to allow passage of the cryo spray, as illustrated in FIG. 8.

Analgesia

It is acceptable to use pharmaceutical analgesia, though the cold temperature of the fluid stream provides analgesic effect. It is acceptable to apply analgesia systemically, topically or even to include analgesic pharmaceuticals suspended in or dissolved in the fluid stream.

Aspiration

It is desirable to aspirate the fluid, the vapor and the skin fragments that are removed by the fluid stream. Preferably a source of suction such as a vacuum pump is provided proximate the area of skin undergoing treatment.

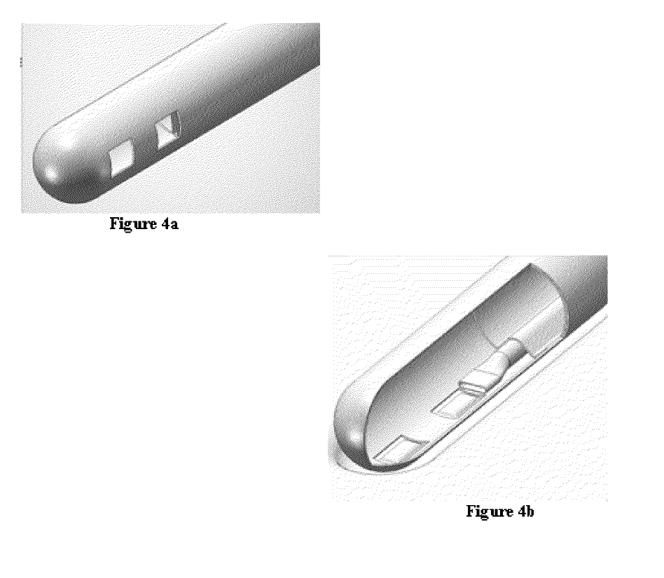

FIG. 4b shows how aspiration can pull the epidermis directly into the path of the injection stream—making it well suited for treatment.

A trap is also preferably provided to collect the liquid and solid debris before it enters the vacuum pump.

FIG. 9 depicts an aspiration embodiment in which a cryo fluid is contained within a pressurized vessel; the flow is metered by a solenoid valve; the fluid pulses interact with the skin which forms the floor of the treatment trough; aspiration channels surrounding the cryo fluid injector nozzle remove skin fragments and cryo fluid.

The most preferred embodiment uses the following elements: an XYZ scanner to move the injector/aspiration instrument tip directly over the skin at a very consistent rate, covering every portion of the target skin uniformly, compressed tetrafluoroethane liquid, solenoid valve controlled 5 millisecond pulses, 100 Hz repetition rate, nozzle that allows air gaps between liquid pulses, tangential pulses directed at skin drawn by half atmosphere vacuum aspiration into the injector path.

When the cold spray is scanned across the skin in the manner described it will resurface the skin; rejuvenating it. As it does that, it also improves the skin appearance by reducing the appearance of age spots, small scars and pox, etcetera. If desired, treatment can be executed to treat focal spots rather than uniformly resurfacing a larger area. In this way, age spots, freckles, warts or other pathologies can be treated so that the appearance of the focal treated spots becomes less evident.

The intensity of the cryo treatment can be adjusted based on the rate at which the cryo fluid is scanned across the surface. If the scanning rate varies (as is common with manual scanning) then the rate at which the cryo spray is delivered to the tissue can be varied automatically by the controller/processor so that the tissue is exposed to a nearly uniform treatment. The intensity of the treatment can be varied by varying pulse rate, pulse time, pulse spray size or equivalent to accommodate variations in scanning.

While the principal application contemplated for the present invention is dermatologic skin resurfacing, many other applications are possible. There is a possible application anywhere there is a need to remove tissue from the body. Examples include cataractous lens removal, tumor resection, liposuction and calculi removal.

It can sometimes be difficult to remove fat in a liposuction procedure without concurrently disrupting blood vessels. This can cause a safety hazard of excessive blood loss. One way this could be avoided is by a technique that differentiates the adipose tissue from the vascular tissue. If one could operate at a temperature and strain rate range at which the fatty tissue is substantially weaker than the vascular tissue, then liposuction could be conducted with less risk of blood loss or other iatrogenic harm. Some techniques have sought to do this at elevated temperature. It may be possible to create better tissue differentiation at cold temperature and high strain rate. The objective is to operate at the conditions where the fat is substantially more brittle and weak than the blood vessels.

Another embodiment could be made which is tailored to function similarly to the fractional skin treatment devices. Instead of using laser radiation, a sharpened needle or electric current to create a narrow-deep lesion, the device of this invention would make the skin brittle to make it more amenable to creating the narrow deep lesion. This would utilize a cold high strain rate coherent stream with a greater boring capacity. This would have a smaller size spray; about 0.1 mm across. It would be desirable to create a higher stress at greater depth compared to the skin resurfacing embodiment. While the cold stream fractional skin treatment device would still preferably use an automatic scanner to create the spaced apart holes, the scanner would remain stationary for intervals as the holes were bored through the skin. Fluid pulses would still be used to attain the high strain rate that makes the tissue more brittle and so easier to “drill” into. Pressurized liquid nitrogen would be a good choice for the medium; the liquid would vaporize shortly after the pulse impact—leaving little but the innocuous nitrogen vapor behind.

In the embodiment where the cold stream is used to facilitate fracture of cataractous lenses, the cold stream can be used independently to fracture pieces of the cataractous lens so that it can be aspirated in small fragments. For the applications of lens and tumors resection, manual control of the movement of the cold spray may be more appropriate than automatic control. While certain aspects of the pulse formation and repetition are suited to some automatic control, manipulation of the location of the erosive spray is better left to the control of the medical practitioner. The cold spray can be used in concert with aspiration to fragment and remove the cataractous lens; or the cold spray can be used in concert with the ultrasound to fragment and remove the lens. Brunescent or dense cataractous can sometimes be difficult to fragment safely and quickly. Use of the cold spray can make these tough lenses more brittle and so easier to fragment. It is important in such circumstances to confine the powerful aspects of the stream to the target tissue. The nozzle should be designed to create an expanding spray, so that the embrittled tissue is limited to the region within the orifice of an aspiration tube—and so neighboring tissue will remain unharmed.

Thus, specific embodiments and applications have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. An apparatus for the treatment of tissue comprising
a source of pressurized fluid having an outlet;
a valve that can be operated to open to allow the flow of the fluid through the outlet and can be closed to stop the flow of the fluid;
the fluid stream having a temperature less than 5° C.
the fluid stream comprised of pulses, said pulses being characterized by a mean pressure, a pressure rise rate and a pressure decay rate.

2. The apparatus of claim 1 further comprising a pressure rise rate or pressure decay rate faster than 100 psi/second.

3. The apparatus of claim 2 further comprising a mean pressure between 10 and 100 psi.

4. The apparatus of claim 1 further comprising a fluid stream that is a mixture of one or more materials chosen from the list containing liquid nitrogen, liquid carbon dioxide, refrigerant R134-A, other refrigerant, a pressurized liquid, analgesic.

5. The apparatus of claim 1 further comprising fluid pulses that contain particulates chosen from the list containing:
particulates that are not solid at room temperature,
particulates that are water soluble,
water soluble material that is not soluble in the fluid,
dry ice,
water ice,
sodium chloride crystals.

6. The apparatus of claim 1 further comprising a scanner to scan the stream across the surface of the tissue.

7. The apparatus of claim 1 further comprising a mechanism that maintains a nearly constant gap between the tissue and the outlet.

8. The apparatus of claim 1 further comprising a scanner that exposes the tissue to a nearly uniform exposure to the stream.

9. The apparatus of claim 1 further comprising a stream chopper.

10. The apparatus of claim 1 further comprising a heater to heat the tissue.

11. The apparatus of claim 1 further comprising at least one aspiration channel, said channel having a proximal end and distal end, said distal end positioned proximate the tissue, said proximal end connected to a source of vacuum pressure to remove fragments and fluid.

12. A method of tissue treatment comprising directing a pressurized cold fluid stream, said stream having a temperature below 5° C., through an injector and out of a distal end of the injector at tissue, wherein the stream causes some fractures in the tissue.

13. The method of claim 12 further comprising scanning the cold fluid stream across a target tissue region.

14. The method of claim 13 further comprising holding the distal end of the injector apart from the tissue surface by a distance less than the distance in which the fluid stream doubles in diameter.

15. The method of claim 12 further comprising directing the longitudinal axis of the stream at an angle chosen from the list containing:
substantially perpendicularly at the tissue surface,
substantially tangentially at the tissue surface,
at a skew angle.

16. The method of claim 12 further comprising changing the temperature of the tissue being treated at a rate greater than 10° C./second.

17. The method of claim 12 further comprising heating the tissue.

18. The method of claim 12 further comprising directing the stream at tissue chosen from the list containing: skin, adipose tissue, subcutaneous fat, a lens.

19. A method of fractional tissue treatment comprising directing a pressurized cold fluid stream through an injector and out of the distal end of the injector at the tissue, wherein the stream creates narrow deep spaced apart lesions.
